(19) United States
(12) Patent Application Publication
(10) Pub. No.: US 2026/0294344 A1
(43) Pub. Date: Oct. 1, 2026

(54) TERAHERTZ SCANNING SYSTEM

(71) Applicant: The University of Warwick, West Midlands (GB)

(72) Inventors: Emma MACPHERSON, West Midlands (GB); Arturo Ignacio HERNANDEZ-SERRANO, West Midlands (GB)

(73) Assignee: The University of Warwick, West Midlands (GB)

(21) Appl. No.: 19/480,707

(22) PCT Filed: Apr. 29, 2024
(86) PCT No.: PCT/GB2024/051134

§ 371 Date: Oct. 31, 2025

(30) Foreign Application Priority Data
May 3, 2023 (GB) 2306553.5

(51) Int. Cl. A61B 5/00; A61B 5/0507

(52) U.S. Cl. CPC A61B 5/6843; A61B 5/0075; A61B 5/0507; A61B 5/444

(57) ABSTRACT

A terahertz scanning system (1₁; 1₂; 1₃) is described. The terahertz scanning system (1₁; 1₂; 1₃) comprises a terahertz source (2) for emitting a terahertz pulse signal (3) into a region under inspection (4) and a terahertz detector (5) for receiving a reflected terahertz pulse signal (6) from the region under inspection (4) and providing a corresponding measurement signal (7). The terahertz scanning system (1₁; 1₂; 1₃) also comprises a contact pressure sensor (9) for determining a contact pressure applied to a surface (11) of the region under inspection (4) and a processing system (8). The processing system (8) is configured, in response to receiving first and second measurement signals: to compare a first measurement signal with a second measurement signal or with a reference measurement signal, the first measurement signal obtained at a first time at which a first contact pressure has been applied for a first contact duration, the second measurement signal obtained at a second time at which a second contact pressure has been applied for a second contact duration; to determine a degree of deviation between the first measurement signal and the second measurement signal or the reference measurement signal; and in dependence on the degree of deviation, to identify a state of the region under inspection (4).

### FIELD

The present invention relates to a terahertz scanning system.

### BACKGROUND

Terahertz (THz) imaging is increasingly being investigated for potential biomedical applications due to its non-ionising nature and strong sensitivity to water content in biological tissue. Terahertz technology has also attracted interest for its ability to identify cancerous and non-cancerous tissues. The water distribution within the upper layers of skin can be caused to change by a change in density or an inability to breathe. Changes such as these in skin and other biological tissues can be detected using commercially available terahertz time domain spectroscopy (THz-TDS) systems.

It is understood that to obtain repeatable results it is necessary to control factors such as skin contact pressure during measurements, for example, as described in H. Lindley-Hatcher et al., “A Robust Protocol for In Vivo THz Skin Measurements”, J. Infrared Millim. Terahertz Waves, vol. 40, pp.980 to 989(2019 ).

Further advantage of controlling factors such as skin contact pressure during measurements can, however, be taken.

### SUMMARY

According to a first aspect of the invention there is provided a terahertz scanning system. The terahertz scanning system includes a terahertz source for emitting a terahertz pulse signal into a region under inspection, a terahertz detector for receiving a reflected terahertz pulse signal from the region under inspection and providing a corresponding measurement signal, a contact pressure sensor for determining a contact pressure applied to a surface of the region under inspection, and a processing system. The processing system is configured, in response to receiving first and second measurement signals, to compare a first measurement signal with a second measurement signal or a reference measurement signal, to determine a degree of deviation between the first measurement signal and the second measurement signal or the reference measurement signal, and, in dependence on the degree of deviation, to identify a state of the region under inspection.

Processing two or more different measurement signals can provide information useful for determining a state of a region under inspection.

The first measurement signal may be obtained at a first time at which a first contact pressure has been applied for a first contact duration, and the second measurement signal may be obtained at a second time at which a second contact pressure has been applied for a second contact duration.

The first contact pressure may be different to the second contact pressure.

The first contact pressure may be applied by exerting a first contact force on a contact area of the surface of the region under inspection. The second contact pressure may be applied by exerting a second contact force on the contact area of the region under inspection.

The first contact force may be equal to or less than 15 N. the second contact force may be equal to or less than 15 N.

The contact area may be equal to or greater than 0.04 cm² and equal to or less than 100 cm² . The area of the surface of the region under inspection may be less than, equal to or greater than the contact area. The contact area may be about 1 cm², about 4 cm², or about 25 cm².

The first contact pressure may be equal to or less than 15 N/cm² . The second contact pressure may be equal to or less than 15 N/cm².

It may be the case that D=|P₂−P₁|, where D is a difference between the second contact pressure, P₂, and the first contact pressure, P₁. The difference may be equal to or greater than 0.1 N/cm² . The difference may be equal to or greater than 0.2 N/cm². The difference may be equal to or greater than 0.5 N/cm² . The difference may be equal to or greater than 1 N/cm² . The difference may be equal to or greater than 2 N/cm².

It may be the case that P_{L}=F×P_{S}, where F is a factor by which the larger of the first and second contact pressures, P_{L}, is larger than the smaller of the first and second contact pressures. The factor may be equal to or greater than 1.05. The factor may be equal to or greater than 1.1. The factor may be equal to or greater than 1.2. The factor may be equal to or greater than 1.5. The factor may be equal to or greater than 2.

The first contact duration may be different to the second contact duration.

The first contact duration may be equal to the second contact duration and the first time may be different to the second time.

The first contact pressure may be lower than the second contact pressure.

The first contact pressure may be zero.

The reference measurement signal may be obtained by measurement of a reference region.

The reference measurement signal may be obtained by simulation of a reference region. The terahertz scanning system may also include an imaging window for applying the contact pressure to the surface of the region under inspection. The imaging window may be arranged between the terahertz source and the region under inspection, and between the region under inspection and the terahertz detector.

The terahertz scanning system may also include a femtosecond pulsed laser for generating a femtosecond laser pulse, a beamsplitter for splitting the femtosecond laser pulse into a first portion and a second portion, and an optical assembly. The optical assembly may be for directing the first portion of the femtosecond laser pulse to the terahertz source, the reflected terahertz pulse signal to the terahertz detector, and the second portion of the femtosecond laser pulse to the terahertz detector via a delay stage.

The terahertz source may include, or take the form of, a photoconductive antenna. The terahertz detector may include, or take the form of, a photoconductive antenna.

The frequency of the terahertz pulse signal may be between about 0.1 THz and about 2 THz.

The terahertz scanning system may be configured to carry out compressed sensing.

The terahertz scanning system may also include a probe comprising the terahertz detector and the contact pressure sensor.

The region under inspection may be a region of a mammalian subject's skin.

The region under inspection may be a region of a mammalian subject's tissue.

The mammalian subject may be a human.

The processing system may be configured to attribute the state to a cosmetic condition.

The state may be a diseased state.

The processing system may be configured to attribute the state to a medical condition.

The processing system may be configured to generate a plan for improving the state.

According to a second aspect of the invention there is provided a method of operating the terahertz scanning system of the first aspect, the method including comparing a first measurement signal with a second measurement signal or a reference measurement signal, determining a degree of deviation between the first measurement signal and the second measurement signal or the reference measurement signal, and, in dependence on the degree of deviation, identifying a state of the region under inspection. The first measurement signal obtained at a first time at which a first contact pressure has been applied for a first contact duration, and the second measurement signal obtained at a second time at which a second contact pressure has been applied for a second contact duration.

According to a third aspect of the invention there is provided a method including comparing a first measurement signal with a second measurement signal or a reference measurement signal, determining a degree of deviation between the first measurement signal and the second measurement signal or the reference measurement signal, and, in dependence on the degree of deviation, identifying a state of the region under inspection. The first measurement signal is obtained at a first time at which a first contact pressure has been applied for a first contact duration, and the second measurement signal obtained at a second time at which a second contact pressure has been applied for a second contact duration.

The method according to the second aspect or the third aspect may include method steps or features corresponding to any features of the terahertz scanning system according to the first aspect.

According to a fourth aspect of the invention there is provided a computer program including instructions which, when executed by a computer, cause the computer to carry out the method according to the second aspect of the invention or the third aspect of the invention.

According to a fifth aspect of the invention there is provided a computer-readable medium storing the computer program according to the fourth aspect of the invention.

### BRIEF DESCRIPTION OF THE DRAWINGS

Certain embodiments of the present invention will now be described, by way of example, with reference to the accompanying drawings in which:

FIG. 1 is a schematic block diagram of a terahertz scanning system;

FIG. 2 is a process flow diagram of a first method of using a terahertz scanning system;

FIG. 3 is a process flow diagram of a second method of using a terahertz scanning system;

FIG. 4 is a process flow diagram of a third method of using a terahertz scanning system;

FIG. 5 is a process flow diagram of a fourth method of using a terahertz scanning system;

FIG. 6 is a more detailed schematic block diagram of a terahertz scanning system; and

FIG. 7 is a detailed schematic block diagram of a terahertz scanning system.

### DETAILED DESCRIPTION OF CERTAIN EMBODIMENTS

In the following, like parts are denoted by like reference numerals.

Herein, it is described that processing two or more different measurement signals can provide information useful for determining a state of a region under inspection.

### Terahertz Scanning System 1₁

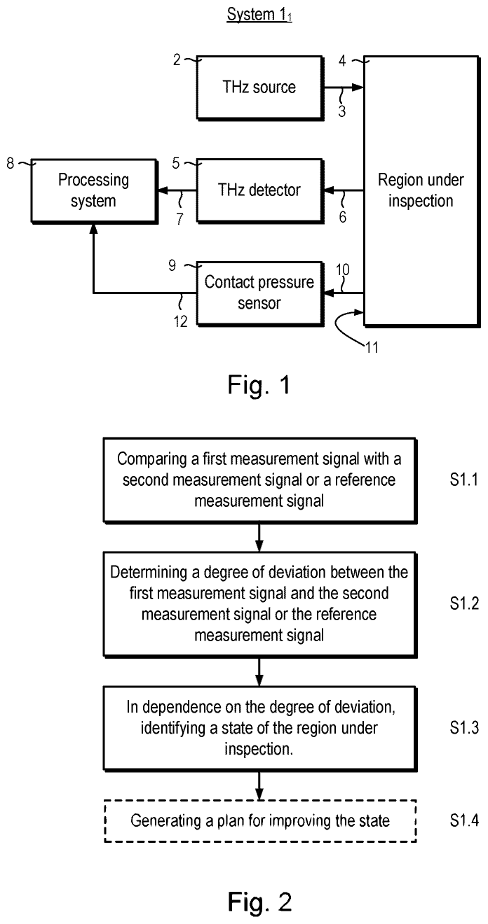

Referring to FIG. 1, a terahertz scanning system 1₁ is shown.

The terahertz scanning system 11 includes a terahertz source 2 for emitting a terahertz pulse 3 into a region under inspection 4. The terahertz scanning system 11 includes a terahertz detector 5 for receiving the reflected terahertz pulse 6 from the region under inspection 4 and providing a corresponding measurement signal 7, and a processing system 8 for processing the received signal 7. The system 1₁ includes a contact pressure sensor 9 which can be brought into mechanical communication 10 with a surface 11 of the region under inspection 4 provide an output signal 12 indicative of the contact pressure applied to the region under inspection 4 to the processing system 8.

The terahertz source 2 and the terahertz detector 5 can each take the form of a photoconductive antenna. The terahertz source 2 may, however, be based on a different suitable operating principle such as surface field emission or optical rectification. Likewise, the terahertz detector 5 may be based on a different operating principle, for example, electro-optical sampling.

A wide variety of sensing modes can be used. The terahertz scanning system 1₁ may carry out compressed sensing measurements. Compressed sensing can gather terahertz image information rapidly without having to raster scan the region under inspection, for example, as described in R. I. Stanchev et al., “Real-time terahertz imaging with a single-pixel detector”, Nat. Commun., vol. 11, pp. 2535(2020 ) which describes using a terahertz compressed sensing fast imaging approach to track hydration changes in a leaf. Whilst known for such purposes, to the applicant's knowledge compressed imaging approaches have not previously been used in examples where the region under inspection 4 is a part of a mammalian subject, let alone a human whose skin or tissue is scanned in vivo. Alternatively, the terahertz scanning system 1₁ may carry out point scan measurements. Point scan measurements can also gather terahertz information rapidly without having to raster scan the region under inspection 4. Due to their rapid nature, compressed sensing and point scan sensing modes can be particularly suitable for measuring time-dependent behaviour of the region under inspection 4.

Output signals 12 provided by the contact pressure sensor 9 to the processing system 8 can enable measurements to be performed in a controlled way and measurement signals 7 obtained from measurements to be comparable, and the terahertz scanning system 11 thereby rendered usable for identifying the state of the region under inspection 4 by way of the configuration of the processing system 8 described herein.

The contact pressure sensor 9 includes one or more pressure sensing elements. A sensing element may take the form of a strain gauge, a piezoelectric pressure sensor, capacitive pressure sensor, or other suitable form of pressure sensor. Including more than one pressure sensing element can improve the accuracy of contact pressure measurements, particularly when these pressure sensing elements are spaced apart. However, including a single pressure sensing element can make the terahertz scanning system 1₁ more compact, cheaper, and simpler to manufacture, and can be useful in cases in which the terahertz detector 5 and the contact pressure sensor 9 are provided in a probe. This is the case particularly when the probe is a hand-held probe. Providing at least the terahertz detector 5 and the contact pressure sensor 9 in a probe can improve ease of measurement, particularly when the probe is a hand-held probe.

### Processing First and Second Measurement Signals Using a Terahertz Scanning System

Referring also to FIG. 2, a first method of using the terahertz scanning system 1₁ will now be described.

In response to receiving first and second measurement signals, the processing system 8 compares a first measurement signal with a second measurement signal or a reference measurement signal (step S1.1), determines a degree of deviation between the first measurement signal and the second measurement signal or the reference measurement signal (step S1.2), and in dependence on the degree of deviation, identifies a state of the region under inspection 4 (step S1.3). The processing system 8 may then generate a plan for improving the state (step S1.4).

The first measurement signal is obtained at a first time at which a first contact pressure has been applied for a first contact duration, and the second measurement signal is obtained at a second time at which a second contact pressure has been applied for a second contact duration. Processing data from first and second (different) measurement signals, or from a measurement signal and a reference measurement signal, can provide information useful for determining the state of the region under inspection 4.

First and second measurement signals provided to the processing system 8 by the terahertz detector 5 take the form of electronic signals, as can the reference measurement signal. Typically, the processing system 8 compares processed versions of these signals. More specifically, the first measurement signal can be compared with the second measurement signal or the reference measurement signal in the time domain (such as peak to peak amplitude, time delay between signals, minimum value in time and so forth) or in the frequency domain (such as spectral amplitude, spectral phase frequency content and so forth). The differences between the compared can be correlated with the physical properties of the region under inspection 4 (such as its reflectivity, refractive index, absorption coefficient, calculated hydration concentration, calculated thickness and so forth). The processing system 8 may, however, compare the first measurement signal with the second measurement signal or a reference measurement signal directly. The reference measurement signal may be obtained by measurement or simulation.

The processing system 8 need not compare individual measurement signals. For example, the processing system can compare two sets of measurement data, with one of those sets including the first measurement signal and the other including the second measurement signal or the reference measurement signal. In the case that sets of measurement data are compared, these may be obtained during the same application of contact pressure, after the same contact duration, or at the same time.

The degree of deviation can be determined in a wide variety of ways. The processing system 8 can evaluate whether a difference between two measurement signals exceeds one or more thresholds, by how much if so, and determine the degree of deviation on that basis (that is, based on how many thresholds are exceeded and/or how much those thresholds are exceeded). In this relatively simple case, the degree of deviation can be described by an integer value. The threshold(s) can relate to the entirety of the difference between the compared measurement signals or one or more parts of this difference, and may be predetermined and/or programmable.

However, the processing system 8 need not determine a degree of deviation using discretely defined thresholds. The threshold(s) may instead take the form of continuous range(s) of values, the proportion of which are exceeded can allow a degree of deviation to be determined which is described by a value which is not discrete.

Further, the threshold(s) can relate to local (that is, specific) parts of the difference between the two measurement signals, instead of its entity. In this case, the degree of deviation can be described by a series of values, or by a curve which is fitted to or connects the series of values.

In the case that the difference falls within the noise level of terahertz scanning system 1₁, the processing system 8 may determine the degree of deviation as being a null value.

The degree of deviation determined can contain a defining characteristic (or “signature”) of a state which can be identified by the processing system 8. The processing system 8 may identify the state by, for example, comparing a determined degree of deviation against a database of degrees of deviation which contain defining characteristics associated with identifiable states. However, the processing system need not identify a state in this way. The terahertz scanning system 11 can compare measurement signals that may be a function of time (for example, an occlusion curve, which is a peak to peak value of an impulse function plotted against duration of measurement) and process relative changes within the measurement signals such as dependencies on skin location, application of a product, a skin condition within the same person, or the vertical location of the occlusion curve relative to occlusion curves from other people.

### Obtaining First and Second Measurement Signals Using the Terahertz Scanning System

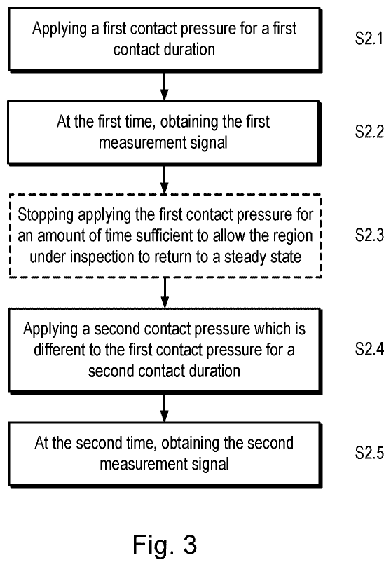

Referring also to FIG. 3, a second method of using the terahertz scanning system 11 will now be described. In the second method, the first and second measurement signals are obtained at different contact pressures.

The second method includes applying a first contact pressure for a first contact duration (step S2.1), at the first time, obtaining the first measurement signal (step S2.2), optionally stopping applying the first contact pressure for an amount of time sufficient to allow the region under inspection 4 to return to a steady state (step S2.3), applying a second contact pressure which is different to the first contact pressure for a second contact duration (step S2.4), and at the second time, obtaining the second measurement signal (step S2.5).

The second method can enable pressure-dependent behaviour of the region under inspection 4 to be used to obtain information for helping to determine its state, particularly when the second contact duration is equal to the first contact duration. Stopping the pressure for a period of time can help to remove the effects of the first contact pressure having previously been applied to the region under inspection 4 by the second time, thus enhancing the comparability of the first and second measurement signals. The second method can be particularly useful when a product such as a moisturiser is applied to the region under inspection 4 after the first measurement signal is obtained but before the second contact pressure is applied (that is, during step S2.3).

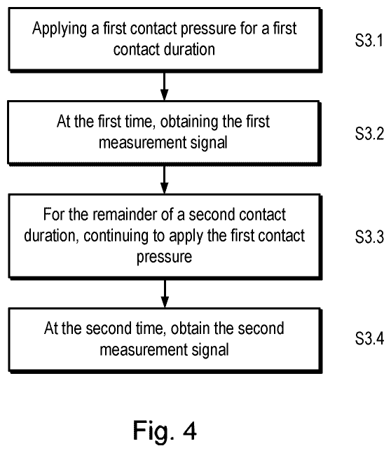

Referring also to FIG. 4, a third method of using the terahertz scanning system 11 will now be described. In the third method, the first and second measurement signals are obtained after different contact durations.

The third method includes applying a first contact pressure for a first contact duration (step S3.1), at the first time, obtaining the first measurement signal (step S3.2), for the remainder of a second contact duration, continuing to apply the first contact pressure (step S3.3), and at the second time, obtaining the second measurement signal (step S3.4).

In the third method, the second contact pressure is equal to the first contact pressure. The third method can enable the time-dependent behaviour of the region under inspection 4 to be used to obtain information for helping to determine its state without needing to apply a second contact pressure which is different to the first contact pressure.

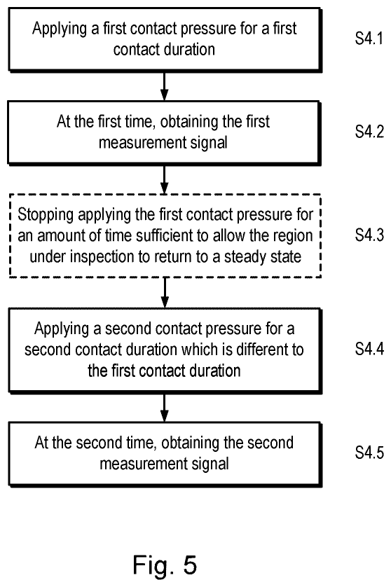

Referring also to FIG. 5, a fourth method of using the terahertz scanning system 1₁ will now be described. In the fourth method, the first and second measurement signals are obtained after equal contact durations and at different times.

The fourth method includes applying a first contact pressure for a first contact duration (step S4.1), at the first time, obtaining the first measurement signal (step S4.2), optionally stopping applying the first contact pressure for an amount of time sufficient to allow the region under inspection to return to a steady state (step S4.3), applying a second contact pressure for a second contact duration which is different to the first contact duration (step S4.4), and at the second time, obtaining the second measurement signal (step S4.5).

The fourth method can enable the time-dependent behaviour of the region under inspection 4 to be used to obtain information for helping to determine its state, particularly when the second contact pressure is equal to the first contact pressure. The fourth method can be particularly useful when a product such as a moisturiser is applied to the region under inspection 4 after the first measurement signal is obtained but before the second contact pressure is applied (that is, during step S4.3).

In each of the second to fourth methods, the terahertz scanning system 1₁ obtains first and second measurement signals during the same application of contact pressure or during separate applications of contact pressure. In the first method, the terahertz scanning system 1₁ uses differences in the pressure and/or time dependent behaviour of the region under inspection 4 to obtain information for helping to determine the state of the region under inspection 4.

In cases in which different contact pressures are to be applied, applying the lower contact pressure first can reduce the amount of time the region under inspection 4 needs to reach a steady state following the first measurement signal being obtained. In cases in which equal contact pressures are to be applied for different contact durations, applying the contact pressure for the lower contact duration before obtaining the first measurement signal can achieve the same effect.

The first contact pressure need not be non-zero. That is, the first measurement signal can correspond to a non-contact measurement in which zero contact pressure is applied in the run up to and during the obtaining of its measurement signal, in contrast to a contact measurement in which a non-zero contact pressure is applied in the run up to and during the obtaining of its measurement signal. In the case that the first measurement signal is from a non-contact measurement, no time is needed for the region under inspection 4 to reach a steady state after the first measurement signal is obtained.

After carrying out any one of the second to fourth methods, the processing system 8 can obtain a third measurement signal corresponding to a third measurement performed after the second measurement signal is obtained. The third measurement signal need not be obtained using the terahertz scanning system 1₁ and may be received from another system. Further, the third measurement may be either a non-contact measurement or a contact measurement.

The region under inspection 4 need not be comprised in the system 1₁.

As hereinbefore described, the region under inspection 4 may be a part of a mammalian subject, such as a human, which is scanned in vivo, and can be, for example, biological tissue, such as skin or the epithelial lining of a colon. In some cases, the region under inspection 4 can be accessed during surgery.

When the region under inspection is a mammalian subject's skin or tissue, using terahertz pulse signals 3 having a frequency between about 0.1 THz and about 2 THz can reduce the level of attenuation, meaning that the reflected terahertz pulse 6 has an amplitude which is detectable using commercially available terahertz time domain spectroscopy (THz-TDS) systems. The frequency of the terahertz pulse signal 3 can, however, be between around 0.1 THz and 5 THz, and the region under inspection 4 need not be a part of a mammalian subject.

### Terahertz Scanning System 1₂

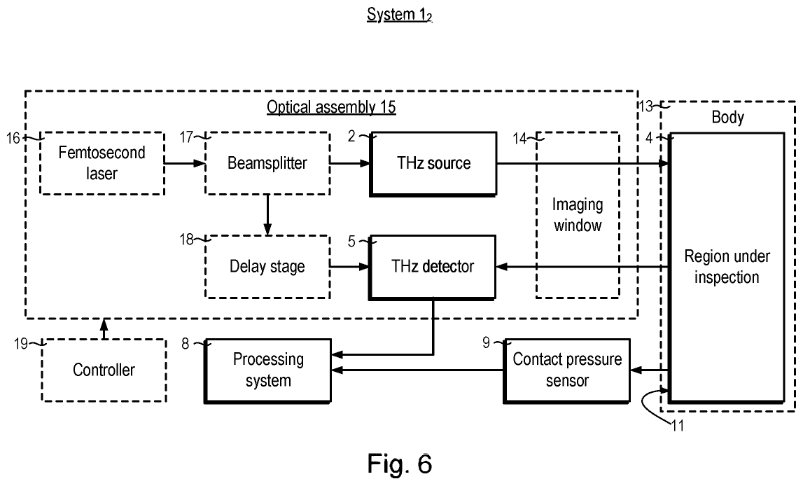

Referring also to FIG. 6, a terahertz scanning system 1₂ is shown. The terahertz scanning system 1₂ is an example of the terahertz scanning system 1₁ and is shown in more detail than the terahertz scanning system 1₁ is in FIG. 1.

Here, the region under inspection 4 is comprised in a body 13 and separated from the terahertz source 2 and terahertz detector by an imaging window 14. The imaging window 14 can be used to apply pressure to the surface 11 of the region under inspection 4 by bringing it into contact with the body 13 or vice versa. The imaging window 14 may be formed from quartz, silicon, or any other suitable material(s).

An optical assembly 15 includes the terahertz source 2, the terahertz detector 5, the imaging window 14, as well as other features concerned with generating the terahertz pulse signal 3 and detecting the reflected terahertz pulse signal 6. The terahertz pulse signal 3 is generated by using a femtosecond pulsed laser 16 to excite the terahertz source 2. The femtosecond pulsed laser 17 may be a titanium-sapphire laser or a mode-locked fibre laser.

A beamsplitter 17 is placed between the femtosecond pulsed laser 16 and the terahertz source 2 and splits the pulse from the femtosecond pulsed laser 16 into first and second portions. The first of these portions is directed to the terahertz source 2 and the second of these portions is directed to the terahertz detector 5 via a delay stage 18. The first and second portions need not have the same intensity. The delay stage is made of a series of mirrors that can be mechanically moved to introduce a time delay between the arrival of the reflected terahertz pulse signal 6 and the arrival of the second portion of the pulse from the femtosecond pulsed laser 16; this can make it possible to sample the reflected terahertz pulse signal 6 across the whole of the time domain window and enable the terahertz scanning system 1₂ to be used to carry out THz-TDS measurements.

A controller 19 can be used to control the optical assembly 15. For example, the pulsing frequency of the laser, the relative intensities of the first and second portions, a DC bias applied across a terahertz source 2 which is a photoconductive antenna, mirror positions and detector position can each be controlled using the controller 19.

In a pulsed mode of operation, the time interval between two consecutively emitted terahertz pulse signals emitted into the region under inspection during a single application of contact pressure corresponds to the pulsing frequency of the femtosecond laser 16. The time interval may be between 10 ms and 1 s, between 100 ms and 500 ms, or around 250 ms. These two consecutively emitted terahertz pulse signals may be included within a burst of similar terahertz pulse signals which spans a period of time between 30 s and 60 s.

Although FIGS. 1 and 6 show a free space system, it is to be understood that the terahertz scanning system 1₂ may be fibre coupled. In particular, optical fibres (not shown) may be used to connect the femtosecond pulsed laser 16 to the terahertz source 2 and the terahertz detector 5.

### Terahertz Scanning System 1₃

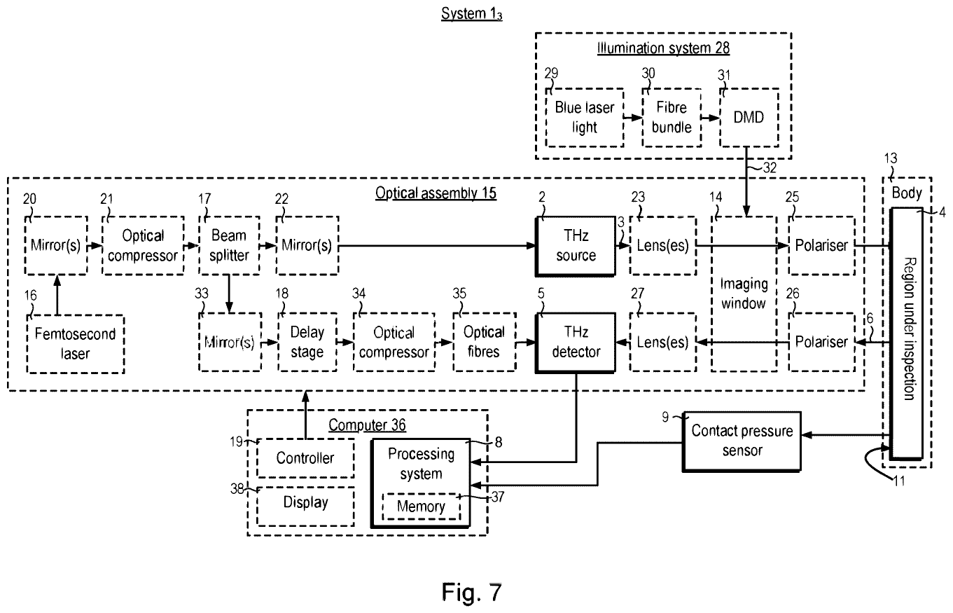

Referring also to FIG. 7, a terahertz scanning system 1₃ is shown in detail. The terahertz scanning system 1₃ is an example of the terahertz scanning system 1₂.

To compensate for dispersion, the pulsed laser light from the femtosecond pulsed laser 16 is guided to the beamsplitter 17 via mirror(s) 20 and an optical compressor 21. Mirror(s) 22 guide the first portion of the pulse from the femtosecond pulsed laser 16 to the terahertz source 2.

On its way from the terahertz source 2 to the region under inspection 4, the terahertz pulse signal 3 passes through lens(es) 23, the imaging window 14, and a polariser 25. Likewise, on its way from the region under inspection 4 to the terahertz detector 5, the reflected terahertz pulse signal 6 passes through polariser 26, the imaging window 14, and lens(es) 27. Using s-polarised light can increase the speed and ease of calculating optical properties from measurement data, for example refractive index. However, there is no need to use s-polarised light. For example, provided that the correct set of equations for processing are used, p-polarised light can be used instead.

An illumination system 28 including a blue laser light 29, fibre bundle 30 and digital micromirror device (DMD) 31 is arranged separately to the optical assembly 15. Modulated blue light 32 from the DMD can be used to illuminate the imaging window 14 to change its optical properties at the same time as the terahertz pulse 3 is incident on the imaging window 14. In this way, the modulated light 32 from the illumination system 28 can cause the energy of a terahertz pulse 3 incident on the region under inspection 4 to change.

Mirror(s) 33 are used to direct the second portion of the pulsed laser light to the delay stage 18. To compensate for dispersion, the pulsed laser light from the delay stage 18 is guided to the terahertz detector 5 via an optical compressor 34 such as a prism compensator and optical fibres(s) 35.

A computer 36 includes the processing system 8 and the controller 19. The processing system 8 has a memory 37 which can store one or more of the first measurement signal, the second measurement signal, and the reference signal. The computer 36 also has a display 38 for displaying information, for example information relating to an identified state. The processing system 8 and/or the controller 19 may, however, be implemented in hardware.

### State Identification

Where the region under inspection 4 is a mammalian subject's skin or a mammalian subject's tissue, the pressure dependence and/or the time-dependence of the first and second measurement signals can correspond to the pressure dependence and/or the time dependence of an occlusion process during measurement of the skin or tissue. This is because the application of different pressures and the passing of time can cause the hydration profile of the skin or tissue being caused to change during measurement, for example by way of occlusion. In this way, the terahertz scanning systems 1₁, 1₂, 1₃ can be used to carry out the first to fourth methods and enable identification of the state of skin or tissue.

When a cosmetic condition such as wrinkles caused by ageing or discolouration due to, for example, sunburn is present on otherwise healthy skin, the processing system 8 can attribute the state to a specific cosmetic condition accordingly.

Likewise, when a medical condition such as skin cancer, psoriasis, skin burn, scar healing or eczema is present (that is, when the state is a diseased state), the processing system 8 can attribute the state to a specific medical condition accordingly. Here, the term diseased state is defined by a particular abnormal condition which negatively affects the normal structure or function of the skin or tissue. In particular, the processing system may attribute the state to an otherwise not visible medical condition, for example a presence of cancer beneath the skin. To accomplish this, terahertz data can be used to calculate the hydration and thickness of the stratum corneum, determine if the skin is thin, thick, dry or hydrated, and whether there is a likely presence of skin cancer based on that determination.

When the no medical or cosmetic condition is present, the processing system 8 can identify that the state is a healthy state.

### Plan Generation

As hereinbefore described, the processing system 8 can generate a plan for improving the state (step S1.4). For example, if a first state is identified, the processing system 8 may look up a corresponding plan or recommendation in a database.

As a simple example, when the region under inspection 4 is an area of dry skin, the measurement signals may reveal a hydration level which is less by a roughly constant amount than a hydration level obtained from measurement of a reference region, and based on the identification of the state as dry skin the processing system 8 may generate a plan for improving the state including, for example, applying a moisturiser, optionally according to a regime that the processing system 8 is able to prescribe based on, for example, a degree of dryness of the dry skin.

### Applications

First and second measurement signals may be used to calculate the hydration and thickness of the stratum corneum and determine if skin is considered to be dry or hydrated. This can be useful to those with dry skin conditions, be used to predict which skin products would best suit a subject, and to assess the effectiveness of various skin products.

Another application relates to patients undergoing therapies which have a side effect to dehydrate the skin, such as radiotherapy. The patient can be measured prior to treatment to determine their pre-treatment skin parameters, and then after treatment any side effects of change in skin hydration can be quantified and a moisturisation strategy planned accordingly, based on the results. The effectiveness can be monitored by regular terahertz measurements.

Yet other applications relate to monitoring wound healing, the effect of transdermal drug delivery patches on the skin and detecting whether a dressing or patch should be removed before it is removed (that is, use of the terahertz scanning system 1₁, 1₂, 1₃ to perform imaging to assess skin through a dressing, a patch, or another skin product).

### Modifications

It will be appreciated that various modifications may be made to the embodiments hereinbefore described. Such modifications may involve equivalent and other features which are already known in the design and use of terahertz scanning systems, and which may be used instead of or in addition to features already described herein. Features of one embodiment may be replaced or supplemented by features of another embodiment.

Although the modes of operation of the terahertz scanning systems 1₁, 1₂, 1₃ described herein are based on pulsed wave imaging, this need not be the case and the mode of operation may instead be based on continuous wave imaging. Continuous wave imaging tends to be cheaper.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A terahertz scanning system, comprising:
a terahertz source for emitting a terahertz pulse signal into a region under inspection;
a terahertz detector for receiving a reflected terahertz pulse signal from the region under inspection and providing a corresponding measurement signal;
a contact pressure sensor for determining a contact pressure applied to a surface of the region under inspection; and
a processing system configured, in response to receiving first and second measurement signals:
to compare a first measurement signal with a second measurement signal or a reference measurement signal;
to determine a degree of deviation between the first measurement signal and the second measurement signal or the reference measurement signal; and
in dependence on the degree of deviation, to identify a state of the region under inspection.

2. The terahertz scanning system of claim 1, wherein:
the first measurement signal obtained at a first time at which a first contact pressure has been applied for a first contact duration; and
the second measurement signal obtained at a second time at which a second contact pressure has been applied for a second contact duration.

3. The terahertz scanning system of claim 2, wherein the first contact pressure is different to the second contact pressure.

4. The terahertz scanning system of claim 2, wherein the first contact duration is different to the second contact duration.

5. The terahertz scanning system of claim 2, wherein the first contact duration is equal to the second contact duration and the first time is different to the second time.

6. The terahertz scanning system of claim 2, wherein the first contact pressure is lower than the second contact pressure.

7. The terahertz scanning system of claim 2, wherein the first contact pressure is zero.

8. The terahertz scanning system of claim 1, wherein the reference measurement signal is obtained by measurement of a reference region.

9. The terahertz scanning system of claim 1, wherein the reference measurement signal is obtained by simulation of a reference region.

10. The terahertz scanning system of claim 1, comprising:
an imaging window for applying the contact pressure to the surface of the region under inspection,
wherein the imaging window is arranged:
between the terahertz source and the region under inspection; and between the region under inspection and the terahertz detector.

11. The terahertz scanning system of claim 1, comprising:
a femtosecond pulsed laser for generating a femtosecond laser pulse;
a beamsplitter for splitting the femtosecond laser pulse into a first portion and a second portion; and
an optical assembly for directing:
the first portion of the femtosecond laser pulse to the terahertz source;
the reflected terahertz pulse signal to the terahertz detector; and
the second portion of the femtosecond laser pulse to the terahertz detector via a delay stage.

12. The terahertz scanning system of claim 1, wherein at least one of the terahertz source and the terahertz detector comprises a photoconductive antenna.

13. The terahertz scanning system of claim 1, wherein a frequency of the terahertz pulse signal is between about 0.1 THz and about 2 THz.

14. The terahertz scanning system of claim 1, wherein the terahertz scanning system is configured to carry out compressed sensing.

15. The terahertz scanning system of claim 1, comprising:
a probe comprising the terahertz detector and the contact pressure sensor.

16. The terahertz scanning system of claim 1, wherein the region under inspection is a region of a mammalian subject's skin.

17. The terahertz scanning system of claim 1, wherein the region under inspection is a region of a mammalian subject's tissue.

18. The terahertz scanning system of claim 16, wherein the mammalian subject is a human.

19. The terahertz scanning system of claim 1, wherein the processing system is configured to attribute the state to a cosmetic condition.

20. The terahertz scanning system of claim 1, wherein the state is a diseased state.

21. The terahertz scanning system of claim 1, wherein the processing system is configured to attribute the state to a medical condition.

22. The terahertz scanning system of claim 1, wherein the processing system is configured to generate a plan for improving the state.

23. A method of operating the terahertz scanning system of claim 1, the method comprising:
comparing a first measurement signal with a second measurement signal or a reference measurement signal, the first measurement signal obtained at a first time at which a first contact pressure has been applied for a first contact duration, the second measurement signal obtained at a second time at which a second contact pressure has been applied for a second contact duration;
determining a degree of deviation between the first measurement signal and the second measurement signal or the reference measurement signal; and
in dependence on the degree of deviation, identifying a state of the region under inspection.

24. A method comprising:
comparing a first measurement signal with a second measurement signal or a reference measurement signal, the first measurement signal obtained at a first time at which a first contact pressure has been applied for a first contact duration, the second measurement signal obtained at a second time at which a second contact pressure has been applied for a second contact duration;
determining a degree of deviation between the first measurement signal and the second measurement signal or the reference measurement signal; and
in dependence on the degree of deviation, identifying a state of a region under inspection.

25. A computer program comprising instructions which, when executed by a computer, cause the computer to carry out the method of claim 23.

26. A computer-readable medium storing the computer program of claim 25.
